(19) United States
(12) Patent Application Publication
(10) Pub. No.: US 2022/0076811 A1
(43) Pub. Date: Mar. 10, 2022

(54) HEALTHCARE CLAIMS SUBMISSION METHOD

(71) Applicants: Ben Manzo, Etobicoke (CA); Nadeen Sara Manzo, Etobicoke (CA)

(72) Inventors: Ben Manzo, Etobicoke (CA); Nadeen Sara Manzo, Etobicoke (CA)

(21) Appl. No.: 17/016,309

(22) Filed: Sep. 9, 2020

(51) Int. Cl. G16H 40/20; G06Q 10/10; G06Q 40/08; G06Q 40/02; G06Q 30/02; G06Q 40/00; G16H 10/20; G06F 16/23

(52) U.S. Cl. CPC G16H 40/20; G06Q 10/10; G06Q 40/08; G06Q 40/02; G06F 16/2379; G06Q 40/10; G06Q 40/123; G06Q 30/0205; G16H 10/20; G06Q 30/0201

(57) ABSTRACT

A method configured to assist a user in the process of submitting a healthcare claim wherein the method of the present invention provides automation thereof. The present invention provides an interface wherein user will engage to register with the service of the present invention. Once registered the service of the present invention will provide monitoring of financial transactions of the individuals banking account information that in order to detect payment to a medical practitioner. Upon detection of payment to a medical practitioner, the operator of the present invention will provide claims submission to the primary insurance provider of the individual. The operator will track the claims submission and provide information thereon to the individual. If necessary, the operator will submit a claim to a secondary insurance provider if a balance remains ensuing payment by the primary insurance provider. Tracking of the individual assists in identifying potential un-submitted claims.

### FIELD OF THE INVENTION

The present invention relates generally to healthcare administration, more specifically but not by way of limitation, an automated healthcare claims submission process that eliminates duplicity of claims processing for a claimant and further provides a plurality of operational efficiencies in the healthcare claims submission process. Furthermore, the present invention will educate the claimant on their healthcare coverage benefits and associated limits including opportunities to obtain financial and/or healthcare benefit related advice tailored to a claimant's own unique circumstances. By way of example but not limitation, a claimant's financial and/or health status as it pertains to a claimant and their familial needs. The present invention will provide direct benefits to the following key stakeholders: claimant, healthcare service provider, insurance provider, claimant's employer and financial advisor.

### BACKGROUND

The healthcare industry is a multi-billion dollar a year industry in North America that is one of the most complex businesses to manage. The combination of patients, healthcare practitioners, healthcare facility providers and insurance companies creates a workflow process that is extremely intricate and tedious. The aforementioned results in a complex administration process that is ultimately a burden not only to all of the providers but also to the patient. Ensuing the receipt of medical services it is typically the patients, also known as claimants, responsibility to initiate the claim for reimbursement for the service. To execute this the claimant will provide information to their primary insurance provider to regarding the service and the fee charged for the service. This can include but not be limited to a physical submission of a receipt or similar document from the healthcare practitioner. It is then the responsibility of the claimant to track each claim while waiting to receive information back from the primary insurance provider regarding the claim submissions.

The claimant will receive an explanation of benefits that will detail the level of reimbursement that the primary insurance provider will provide for the service rendered based on the claimant's current plan coverage. This reimbursement can range from no reimbursement to full reimbursement. The individual claimant must locate the explanation of benefits on the primary insurance providers website and as such spends time monitoring for the publishing thereof. If the claimant receives no or partial reimbursement for the claim and the claimant is either married, living common-law, and/or responsible for household dependents, the claimant can submit another claim to their spouse's (or partner's) primary insurance provider to be reimbursed for any residual balance. During submission of a second claim to a spouse's primary insurance provider the claimant is subjected to a significant amount of redundancy in the submission process wherein the claimant will have to provide the same information, answer the same questions and submit the same receipt evidence as was executed for the original submission for the claim. Furthermore, the claimant must additionally keep a historical summary of all submissions, amounts paid, amounts reimbursed and not reimbursed for tax preparation and submissions in order to ensure they have been reimbursed for all services rendered according to insurance plan coverages. The aforementioned is complicated, time consuming and inefficient.

It is intended within the scope of the present invention to provide a healthcare claims submission process that eliminates redundancy, leverages information sharing and drives operational efficiency in healthcare claim submission processes.

### SUMMARY OF THE INVENTION

It is the object of the present invention to provide a method for healthcare claims submission that is operable to provide operational efficiencies and redundancy elimination wherein the present invention includes claimant registration via a website that collects and will link pertinent information for subsequent utilization.

Another object of the present invention is to provide a healthcare claims submission method configured to provide automation within the process of submitting a claim to at least one insurance provider wherein the present invention provides automated notification of a rendered healthcare service to the primary insurance provider ensuing receipt of a healthcare service.

A further object of the present invention is to provide a method for healthcare claims submission that is operable to provide operational efficiencies and redundancy elimination wherein the present invention will provide a summary of the claim to the claimant.

Still another object of the present invention is to provide a healthcare claims submission method configured to provide automation within the process of submitting a claim to at least one insurance provider wherein the present invention provides tracking of balances paid and any residual balances during the claims submission process.

An additional object of the present invention is to provide a method for healthcare claims submission that is operable to provide operational efficiencies and redundancy elimination wherein the method of the present invention further includes obtaining, storing and providing access to explanation of benefit summaries.

Yet a further object of the present invention is to provide a healthcare claims submission method configured to provide automation within the process of submitting a claim to at least one insurance provider wherein in the event of a residual balance to the claimants initial primary claim the present invention will provide automated submission to a second primary insurance provider.

Another object of the present invention is to a provide a method for healthcare claims submission that is operable to provide operational efficiencies and redundancy elimination wherein the method further included automated generation of historical unpaid balances for income tax submission.

An alternate object of the present invention is to provide a healthcare claims submission method configured to provide automation within the process of submitting a claim to at least one insurance provider wherein the present invention can further provide tracking of healthcare providers visited during a certain period of time so as to cross-reference with filed claims.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a flowchart of the process facilitated by the present invention.

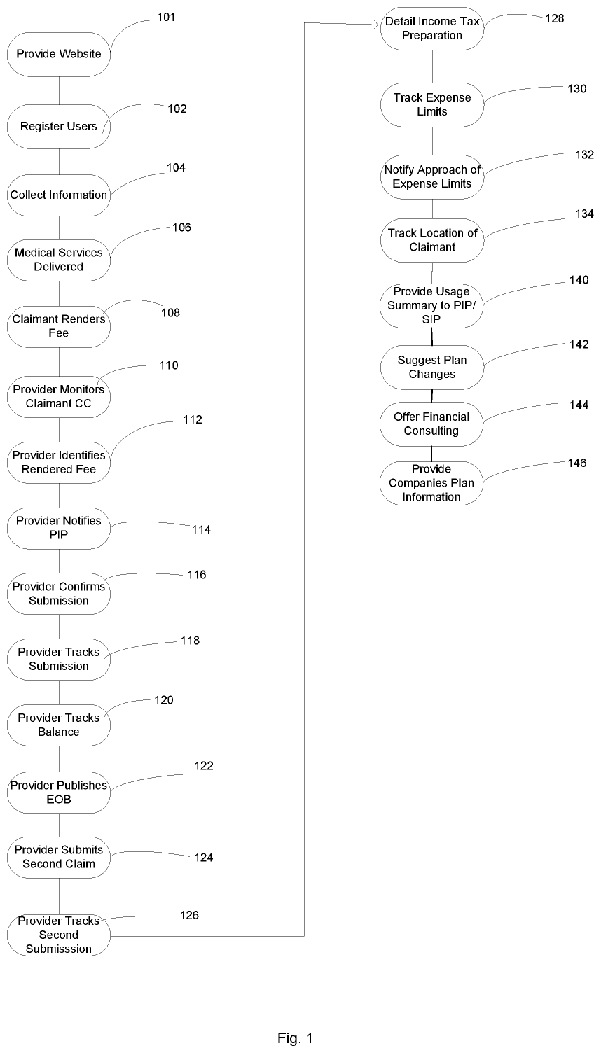

### DETAILED DESCRIPTION

Referring now to the drawings submitted herewith, wherein various elements depicted therein are not necessarily drawn to scale and wherein through the views and figures like elements are referenced with identical reference numerals, there is illustrated a healthcare claims submission method 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Now referring in particular to the Figure submitted as a part hereof, the healthcare claims submission method 100 is provided via an operational interface of a website in step 101. It should be understood within the scope of the present invention that the method thereof is provided utilizing conventional computing devices such as but not limited to personal computers, tablet PCs and/or smart phones wherein the aforementioned are utilized to access a website that has been constructed and provided by the operator of the healthcare claims submission method 100. The graphical interface of the website of the present invention is a conventional graphical interface that a user can navigate to execute the desired functions of the present invention. It should be further understood within the scope of the present invention that present invention could be executed via a software application either in place of and/or in conjunction with a website. Step 102 comprises of the operator soliciting and registering a plurality of users. It should be understood that the user are the following: claimant, healthcare service provider, insurance provider, claimant's employer and financial advisor. In step 104, ensuing solicitation of users and during the registration process, the operator will collect from each user a specific parameter set of information that is required in order to facilitate execution of the healthcare claims submission method 100. More specifically but not by way of limitation, the operator will collect the following: personal demographic information to include but not be limited to name, address and contact information, employer information, primary health insurance company provider, primary banking information to include such items as but not limited to credit card information and a voided check and identities of medical service providers routinely utilized by the registering user. Furthermore, the registering user will be asked to enable tracking location on their smart phone wherein as is further discussed herein will be utilized to cross reference locations visited and healthcare claims filed.

In step 106, a registered user will visit and receive services from a medical practitioner. It should be understood within the scope of the present invention that the medical practitioner can include but is not limited to any type of medicine and/or dental services. Step 108, ensuing delivery of the medical services, the registered user will utilize a form of payment that was captured during the registration process with the operator of the healthcare claims submission method 100. In step 110, the operator of the healthcare claims submission method 100 provides continuous monitoring of the registered users credit card and other forms of payment that were captured during the registration process of the user. Monitoring of the payment forms is specifically focused on any provider that is in the healthcare industry such as but not limited to a doctor or a dentist. Step 112, during the monitoring process the operator of the healthcare claims submission method 100 detects a fee rendered to a medical practitioner. In step 114, the operator of the healthcare claims submission method 100 will provide automatic notification to the registered users primary insurance provider for the registered user of the fee rendered to the medical practitioner. Step 116, during this process a summary of the claim and pertinent information thereof will be sent to the registered user along with confirmation that the primary insurance provider has been notified of the rendered payment.

In step 118, the operator of the healthcare claims submission method 100 provides submission of the explanation of benefits generated by the primary insurance provider to the registered user and additionally in step 120 the operator will track the balances paid and due for the medical service rendered. It should be understood within the scope of the present invention that the medical practitioner will not have to provide a receipt of services as the documentation provided by the operator of the healthcare claims submission method 100 will be a substitute therefore. The medical practitioner registers with the healthcare claims submission method 100 in order to receive the following additional benefits: no merchant devices needed for claimants also registered with the present invention resulting in no merchant fees incurred resulting in less paper trail and more automation, receipt of services rendered provided to claimant upon confirmation from the medical and/or dental service provider, option for service provider to opt for direct communication with a claimant, i.e. questions about historical services rendered, exercises to complete, book medical appointments. Further benefits include calendar sharing—the healthcare claims submission method 100 will provide a claimant and the medical practitioner access calendar availability to schedule future appointments, access to a claimant's history of previous services rendered with a service provider to highlight opportunities for additional treatment when applicable and/or necessary, automated audit history per claimant for the medical practitioner to use for accounting and tax filing and be registered as a medical practitioner registered with the healthcare claims submission method 100 for claimants looking for a medical pracititioner.

In step 122, the operator of the healthcare claims submission method 100 will retrieve and provide automatically to the registered user the explanation of benefits for the rendered medical service. Furthermore, the healthcare claims submission method 100 provides real time access to coverage types, balances, accessibility of local medical practitioners, and further provide the ability to book an appointment for a medical practitioner when required for instances such as but not limited to an annual physical.

In step 124, if the registered user's primary insurance provider does not provide full payment of the fee rendered to by the medical practitioner, the healthcare claims submission method 100 provides detection thereof and automatically triggers a payment reimbursement request to a secondary insurance provider. Additionally, the explanation of benefits and receipts of services rendered are submitted along with the explanation of benefits and receipt of service(s) rendered. Step 126, the operator of the healthcare claims submission method 100 provides tracking of the submission to the secondary insurance provider and will further publish to make available to the registered user. During steps 124 and 126, the healthcare claims submission method 100 will provide automatic notification to the registered user that a remaining balance exists for the rendered medical service subsequent submission to the primary insurance provider and that a claim has been submitted to the registered user's secondary insurance provider. The operator of the healthcare claims submission method 100 will provide notification of the remaining balance that was submitted and whether or not it's been reimbursed. If not, the claimant(registered user) will be notified of the unpaid balance to be used for tax filing. If not applicable the operator of the healthcare claims submission method 100 will provide notification of the remaining balance that will be billed to the registered user's form of payment.

In step 128, the healthcare claims submission method 100 provides detailed medical tax credit information and further provide the ability to generate the necessary statements representing unpaid balances to help in the submission of taxes. In step 130, throughout the entire process the healthcare claims submission method 100 will track expense limits pertinent to the registered user and their healthcare plan provided by their primary and/or secondary insurance provider. Step 132, upon becoming proximate to the aforementioned expense limits the registered user will receive an automated notification from the healthcare claims submission method 100. In step 134, as previously mentioned herein, the healthcare claims submission method 100 provides geo-location tracking for those registered users that have opted in for this feature. The geo-location tracking of the registered user is configured to cross reference the user's location over a time period such as but not limited to twelve months with the geo-location of medical practitioners. If during the cross referencing of the geo-locations of the registered user and medical practitioners the operator of the healthcare claims submission method 100 identifies that the registered user was at a geo-location of a medical practitioner and no record of a claim exists for that practitioner, a notification will be sent to the registered user inquiring whether or not the registered user received a medical service from the identified practitioner and is so to submit a claim therefore.

In step 140, the healthcare claims submission method 100 further functions to provide usage summaries to the primary insurance providers of the registered users. In step 142, the providing of this report offers the ability for a primary insurance provider to utilize the registered user's history and provide offers of alternative coverage's that may be more effective for the registered user. This can assist the registered user with budgeting, benefit planning and annual enrollment. Step 144, the healthcare claims submission method 100 further provides the registered user with an ability to liaise with a financial consultant so as to offer further counseling on healthcare plan coverage's and financial planning. In step 146, the healthcare claims submission method 100 is further configured to provide recommendations to companies on the benefit levels of healthcare plans to offer their employees that would be operable to drive parameters such as but not limited to employee satisfaction. The healthcare claims submission method 100 executes the aforementioned by consulting with the primary insurance providers and businesses to expand and/or optimize the level of healthcare coverage. Additionally, the healthcare claims submission method 100 is configured to provide an incentive to businesses for those businesses who have a certain percentage of employees register with the healthcare claims submission method 100.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. A method for providing healthcare claims submission and tracking thereof for an individual comprising the steps of:
providing a user interface, wherein the user interface is configured to offer a graphical engagement of the method for providing healthcare claims submission;
registering a multitude of individual users, wherein the individual users utilize the user interface to register with an operator of the method for providing healthcare claims submission;
collecting information from the multitude of individual users, wherein the information includes personal demographic information, banking account information and primary insurance provider information;
monitoring the banking account information of the multitude of individuals for purchase of a medical service, wherein the operator of the method for providing healthcare claims submission monitors the banking account information of the multitude of individuals;
notifying the primary insurance provider of one of the multitude of individuals that a medical service has been purchased, wherein a claim for the purchase is submitted;
confirming submission of the notification to the primary insurance provider, wherein the operator provides confirmation to an individual that a claim has been submitted to the primary insurance provider;
checking for publication of an explanation of benefits, wherein the operator of the method for providing healthcare claims submission will check for an explanation of benefits;
notifying the multitude of individuals of expense limits, wherein the operator of the method for providing healthcare claims submission notifies individuals if an expense limit is being approached; and
providing claim amount relevant for income tax filing, wherein the operator will provide the multitude of individuals with data regarding any amount of a medical service that is relevant for tax filing.

2. The method for providing healthcare claims submissions as recited in claim 1, and further including the step of tracking a geo-location of the multitude of individuals, wherein the geo-location of the multitude of individuals is tracked and recorded by the operator of the method for providing healthcare claims submissions.

3. The method for providing healthcare claims submissions as recited in claim 2, and further including the step of cross referencing the geo-locations of the individuals with a database of medical service providers.

4. The method for providing healthcare claims submissions as recited in claim 3, and further including the step of notifying a secondary insurance provider, wherein the secondary insurance provider is notified in an event of non-payment or partial payment by the primary insurance provider.

5. The method for providing healthcare claims submissions as recited in claim 4, and further including the step of monitoring for an explanation of benefits from the secondary insurance provider.

6. The method for providing healthcare claims submissions as recited in claim 5, and further including the step of suggesting plan coverage changes to the multitude of individuals registered with the method for providing healthcare claims submissions based on historical purchased medical services.

7. The method for providing healthcare claims submissions as recited in claim 6, and further including the step of tracking a balance of available benefits from the primary insurance provider, wherein the available balance is transmitted to the multitude of individuals.

8. A method for providing healthcare claims submission and tracking thereof for multitude of individuals wherein the method comprises the steps of:
providing a user interface, wherein the user interface is a graphical interface on a computing device being configured to provide operation of the method for providing healthcare claims submission;
registering a multitude of individual users, wherein the multitude of individual users utilize the user interface to register with an operator of the method for providing healthcare claims submission;
collecting information from the multitude of individual users, wherein the information includes personal demographic information, banking account information, identities of medical service providers routinely utilized by the multitude of individuals and primary insurance provider information;
receiving a medical service from a medical practitioner, wherein at least one of the multitude of individuals receives a medical service;
rendering payment for the medical service, wherein the at least one of the multitude of individuals renders payment to the medical practitioner;
monitoring the banking account information of the multitude of individuals for purchase of a medical service, wherein the operator of the method for providing healthcare claims submission monitors the banking account information of the multitude of individuals;
notifying the primary insurance provider of one of the multitude of individuals that a medical service has been purchased, wherein a claim for the purchase is submitted;
confirming submission of the notification to the primary insurance provider, wherein the operator provides confirmation to an individual that a claim has been submitted to the primary insurance provider;
checking for publication of an explanation of benefits, wherein the operator of the method for providing healthcare claims submission will check for an explanation of benefits;
providing the explanation of benefits to the individual;
submitting an additional claim to a secondary insurance provider, to include an explanation of benefits and a receipt for service(s) rendered and wherein the operator of the method for providing healthcare claims submission submits a claim if a balance remains for the medical service subsequent submission to the primary insurance provider; and
providing claim amount relevant for income tax filing, wherein the operator will provide the multitude of individuals with data regarding any amount of a medical service that is relevant for tax filing.

9. The method for providing healthcare claims submission and tracking thereof as recited in claim 8, and further including the step of notifying the multitude of individuals of expense limits, wherein the operator of the method for providing healthcare claims submission notifies individuals if an expense limit is being approached.

10. The method for providing healthcare claims submission and tracking thereof as recited in claim 9, and further including the step of tracking a geo-location of the multitude of individuals, wherein the geo-location of the multitude of individuals is tracked and recorded by the operator of the method for providing healthcare claims submissions.

11. The method for providing healthcare claims submission and tracking thereof as recited in claim 10, and further including the step of cross referencing the geo-locations of the individuals with a database of medical service providers.

12. The method for providing healthcare claims submission and tracking thereof as recited in claim 11, and further including the step of contacting the multitude of individuals to confirm if a medical practitioner was visited and a service rendered upon failure to identify payment in the banking account information.

13. The method for providing healthcare claims submission and tracking thereof as recited in claim 12, and further including the step of suggesting plan coverage changes to the multitude of individuals registered with the method for providing healthcare claims submissions based on historical purchased medical services.

14. The method for providing healthcare claims submission and tracking thereof as recited in claim 13, and further including the step of providing claim amounts relevant for income tax filing, wherein the operator will track unpaid balances by the primary and secondary insurance provider and provide the multitude of individuals with data regarding any amount of a medical service that is relevant for tax filing.

15. A method for providing healthcare claims submission and tracking thereof for multitude of individuals wherein the method comprises the steps of:
providing a user interface, wherein the user interface is a graphical interface on a computing device being configured to provide operation of the method for providing healthcare claims submission;
registering a multitude of individual users, wherein the multitude of individual users utilize the user interface to register with an operator of the method for providing healthcare claims submission;
collecting information from the multitude of individual users, wherein the information includes personal demographic information, banking account information, identities of medical service providers routinely utilized by the multitude of individuals and primary insurance provider information;
receiving a medical service from a medical practitioner, wherein at least one of the multitude of individuals receives a medical service;
rendering payment for the medical service, wherein the at least one of the multitude of individuals renders payment to the medical practitioner;
monitoring the banking account information of the multitude of individuals for purchase of a medical service, wherein the operator of the method for providing healthcare claims submission monitors the banking account information of the multitude of individuals;
tracking a geo-location of the multitude of individuals, wherein the geo-location of the multitude of individuals is tracked and recorded by the operator of the method for providing healthcare claims submissions;
referencing the geo-locations of the individuals with a database of geo-locations of medical service providers;
identifying that an individual was present at a geo-location of a medical service provider and no record of payment to the medical service provider exists in the banking account information;
notifying the primary insurance provider of one of the multitude of individuals that a medical service has been purchased, wherein a claim for the purchase is submitted;
confirming submission of the notification to the primary insurance provider, wherein the operator provides confirmation to an individual that a claim has been submitted to the primary insurance provider;
checking for publication of an explanation of benefits, wherein the operator of the method for providing healthcare claims submission will check for an explanation of benefits;
providing the explanation of benefits to the individual;
submitting an additional claim to a secondary insurance provider, wherein the operator of the method for providing healthcare claims submission submits a claim if a balance remains for the medical service subsequent submission to the primary insurance provider; and
providing claim amount relevant for income tax filing, wherein the operator will provide the multitude of individuals with data regarding any amount of a medical service that is relevant for tax filing.

16. The method for providing healthcare claims submission and tracking thereof as recited in claim 15, and further including the step of contacting the multitude of individuals to confirm if a medical practitioner was visited and a service rendered upon failure to identify payment in the banking account information.

17. The method for providing healthcare claims submission and tracking thereof as recited in claim 16, and further including the step of providing claim amounts relevant for income tax filing, wherein the operator will track unpaid balances by the primary and secondary insurance provider and provide the multitude of individuals with data regarding any amount of a medical service that is relevant for tax filing.

18. The method for providing healthcare claims submission and tracking thereof as recited in claim 17, and further including the step of suggesting plan coverage changes to the multitude of individuals registered with the method for providing healthcare claims submissions based on historical purchased medical services.

19. The method for providing healthcare claims submission and tracking thereof as recited in claim 18, and further including the step of tracking a balance of available benefits from the primary insurance provider and the secondary insurance provider, wherein the available balance is transmitted to the multitude of individuals.

20. The method for providing healthcare claims submission and tracking thereof as recited in claim 19, and further including the step of providing healthcare plan information to companies, wherein the plan information contains information about alternate plans for the multitude of individuals.
